(19) United States
(12) Patent Application Publication
(10) Pub. No.: US 2022/0330847 A1
(43) Pub. Date: Oct. 20, 2022

(54) Detection of Tissue Damage from Personal Protective Equipment

(71) Applicant: Bruin Biometrics, LLC, Los Angeles, CA (US)

(72) Inventors: Martin F. BURNS, Los Angeles, CA (US); Vignesh Mani IYER, Torrance, CA (US)

(73) Assignee: BRUIN BIOMETRICS, LLC, Los Angeles, CA (US)

(21) Appl. No.: 17/724,668

(22) Filed: Apr. 20, 2022

Related U.S. Application Data
(60) Provisional application No. 63/177,132, filed on Apr. 20, 2021.
(60) Provisional application No. 63/332,353, filed on Apr. 19, 2022.

(51) Int. Cl. A61B 5/0537; A41D 13/11; A61B 5/0531

(52) U.S. Cl. CPC A61B 5/0537; A41D 13/1161; A61B 5/0531; A41D 13/1184

(57) ABSTRACT

Methods and apparatus for detection of tissue damage in users using a PPE device for an extended period of time are disclosed.

### CROSS-REFERENCE TO RELATED APPLICATIONS

This application claims priority to U.S. Provisional Application No. 63/177,132 filed Apr. 20, 2021, and U.S. Provisional Application No. 63/332,353 filed Apr. 19, 2022, the contents of each of which are hereby incorporated by reference in their entirety.

### FIELD

The present disclosure provides methods and apparatus for detecting tissue damage resulting from personal protective equipment, through measurement of Sub-Epidermal Moisture (SEM) and evaluation of those measurements.

### BACKGROUND

The skin is the largest organ in the human body. It is readily exposed to different kinds of damages and injuries. When the skin and its surrounding tissues are unable to redistribute external pressure and mechanical forces, ulcers may be formed. Prolonged continuous exposure to even modest pressure, such as the pressure created by frequent wearing of personal protective equipment (PPE) on skin surfaces, may lead to a pressure ulcer.

Healthcare professionals and users in acute or long-term care facilities, may be required to wear personal protective equipment, like N-95 masks, googles, and face shields, for an extended period of time. Some PPE devices are in contact with portions of the user's body, for example, an N95 mask that is in contact with the nose, cheeks, and chin. Mask materials may mechanically indent and damage facial skin, an effect that is further compromised by perspiration. This puts users of PPE at greater risk for PPE device-related pressure injuries. The long-term pressure applied by these devices may be low but the extended period of application may lead to tissue damage that, left untreated, may progress to an open ulcer.

### SUMMARY

In an aspect, the present disclosure provides for, and includes, an apparatus for detecting tissue damage proximate to a point of contact between a PPE device and a user's skin, comprising: a first electrode and a second electrode configured to measure a level of sub-epidermal moisture (SEM) in tissue proximate to the point of contact, an electronics package individually connected to the first and second electrodes and configured to measure a capacitance between the first and second electrodes.

In an aspect, the present disclosure provides for, and includes, a method for detecting tissue damage proximate to a point of contact between a PPE device and a user's skin, comprising the steps of: measuring a plurality of sub-epidermal moisture (SEM) values of tissue proximate to the point of contact at incremental times, comparing the plurality of SEM values, and determining if there is a significant increase in the SEM that indicates that there is tissue damage.

### BRIEF DESCRIPTION OF THE DRAWINGS

Aspects of the disclosure are herein described, by way of example only, with reference to the accompanying drawings. With specific reference now to the drawings in detail, it is stressed that the particulars shown are by way of example and are for purposes of illustrative discussion of aspects of the disclosure. In this regard, the description and the drawings, considered alone and together, make apparent to those skilled in the art how aspects of the disclosure may be practiced.

FIG. 1 depicts a user wearing an N95 mask.

FIG. 2 depicts a user wearing a valved respirator.

FIG. 3A illustrates the pressure-induced damage associated with a diagnosis of a stage-1 pressure ulcer.

FIG. 3B depicts a user who has developed a pressure ulcer from a PPE device on his face.

FIG. 4A depicts a user wearing a PPE device with a Sub-Epidermal Moisture (SEM) sensor, in accordance with the present disclosure.

FIG. 4B depicts a SEM sensing system, in accordance with the present disclosure.

FIG. 4C depicts a SEM sensing system, in accordance with the present disclosure.

FIG. 5A illustrates how a PPE device may contact a user.

FIG. 5B depicts a SEM sensing device, in accordance with the present disclosure.

FIG. 5C is an enlarged view of a portion of the device of FIG. 5B, in accordance with the present disclosure.

FIG. 6A depicts a user wearing a PPE device that incorporates a forehead band, in accordance with the present disclosure.

FIG. 6B is a view of the underside of the forehead band of FIG. 6A, in accordance with the present disclosure.

FIGS. 7A and 7B depict example PPE devices with controllable pressure management elements, in accordance with the present disclosure.

FIG. 8 depicts an N95 fast mask with controllable pressure management elements, in accordance with the present disclosure.

### DETAILED DESCRIPTION

This description is not intended to be a detailed catalog of all the different ways in which the disclosure may be implemented, or all the features that may be added to the instant disclosure. For example, features illustrated with respect to one embodiment may be incorporated into other embodiments, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces, and processes have not been shown in detail in order not to unnecessarily obscure the invention. It is intended that no part of this specification be construed to effect a disavowal of any part of the full scope of the invention. Hence, the following descriptions are intended to illustrate some particular embodiments of the disclosure, and not to exhaustively specify all permutations, combinations, and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular aspects or embodiments only and is not intended to be limiting of the disclosure.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

U.S. patent application Ser. No. 14/827,375 discloses an apparatus that measures the sub-epidermal capacitance using a bipolar sensor, where the sub-epidermal capacitance corresponds to the moisture content of the target region of skin of a user. The '375 application also discloses an array of these bipolar sensors of various sizes.

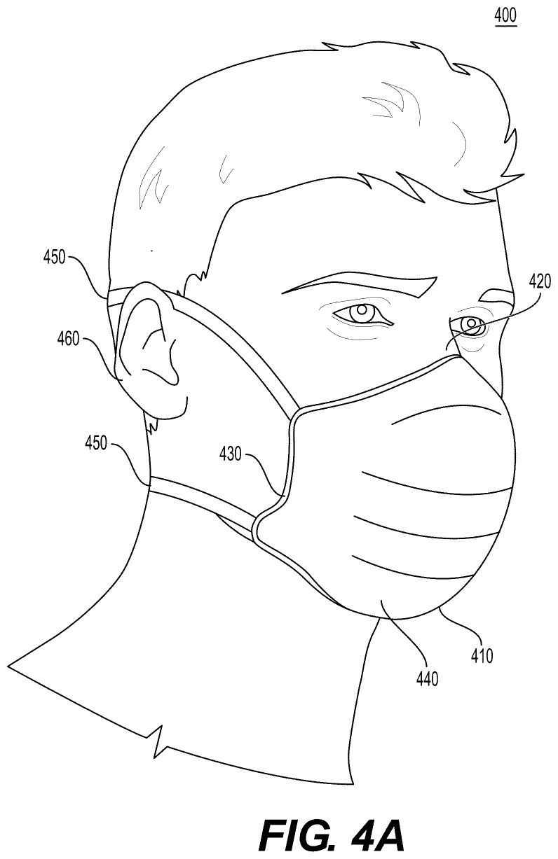

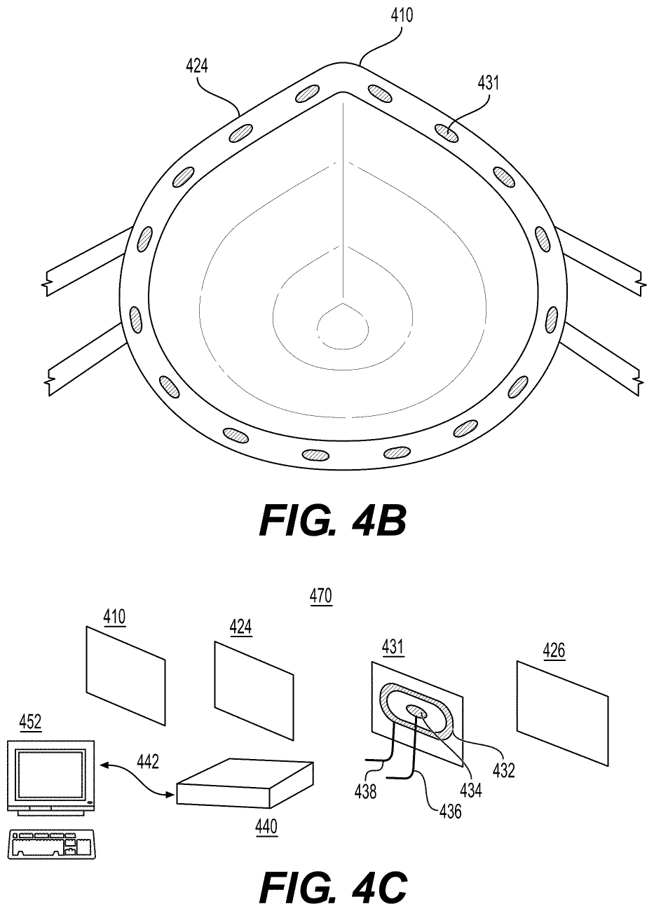

U.S. Pat. No. 10,182,740B2 discloses an apparatus for measuring sub-epidermal moisture (SEM) similar to the device shown in FIG. 4C, where the device emits and receives an RF signal at a frequency of 32 kHz through a single coaxial sensor and generates a bioimpedance signal, then converts this signal to a SEM value.

Both U.S. Pat. Nos. 9,398,879B2 and 10,182,740B2 are incorporated herein by reference in their entireties.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

The methods disclosed herein include and comprise one or more steps or actions for achieving the described method. The method steps and/or actions may be interchanged with one another without departing from the scope of the present disclosure. In other words, unless a specific order of steps or actions is required for proper operation of the embodiment, the order and/or use of specific steps and/or actions may be modified without departing from the scope of the present disclosure.

As used in the description of the disclosure and the appended claims, the singular forms “a,” “an,” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a length, a frequency, or a SEM value and the like, is meant to encompass variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

As used herein, the term “sub-epidermal moisture” or “SEM” refers to the increase in tissue fluid and local edema caused by vascular leakiness and other changes that modify the underlying structure of the damaged tissue in the presence of continued pressure on tissue, apoptosis, necrosis, and the inflammatory process.

As used herein, a “user” may be a human or animal subject.

As used herein, “delta” refers to a calculated difference between two SEM values.

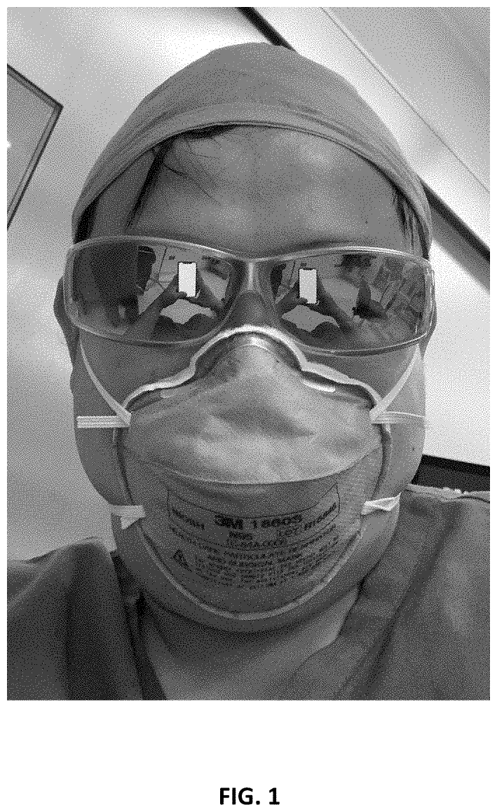

FIG. 1 depicts a user 100 wearing an N95 mask 110. An N95 mask is used by individuals requiring particulate filtering for respiration, including, but not limited to workers in mines, construction, and industrial settings. An N95 mask may also be used when visiting a toilet. An N95 may also be used by healthcare workers in prevention of infectious air-borne diseases. The N95 mask 110 is worn at the workplace, or in the presence of potential contaminants, for typically 8-12 hour shifts or longer. This repeated exposure of sensitive facial tissue, where the skin is close to bone (bony prominence), to long-duration low-pressure contact at the bridge of nose 120, cheeks 130, and chin 140 poses a risk of developing a pressure ulcer.

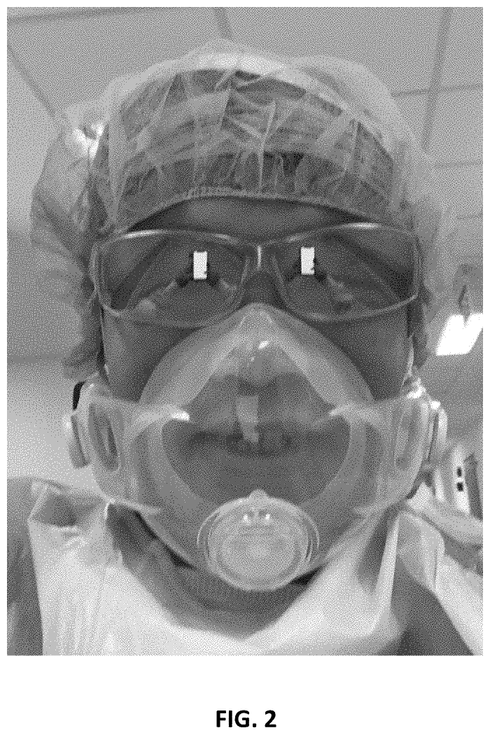

FIG. 2 depicts a user 200 wearing a safety goggles 210 and valved respirator 220. Safety goggles 210 may be worn to shield from heat and splash hazards. A valved respirator 220 may be worn in a healthcare setting to prevent contamination from viruses or bacteria aerosolized from coughing and sneezing. Without being bound by theory, aerosol-generating procedures in a healthcare setting are procedures performed on patients that are more likely to generate higher concentrations of infectious respiratory aerosols than coughing, sneezing, talking, or breathing. In an aspect, PPE devices are worn during aerosol-generating procedures that include, but are not limited to, open suctioning of airways, sputum induction, manual ventilation, endotracheal intubation and extubation, noninvasive ventilation, bronchoscopy, and tracheotomy. Without being bound by theory, toilet aerosol can also be a vector for diseases with the shedding of large numbers of pathogens through feces, vomit and other bodily fluids. In an aspect, PPE devices are worn when the toilet is used or flushed. The valved respirator 220 is worn at the workplace, or in the presence of potential contaminants, for typically 8-12 hour shifts or longer. This repeated exposure of sensitive facial tissue, where the skin is close to bone (bony prominence), to long-duration low-pressure contact at the bridge of nose, cheeks, chin, and ears poses a risk of developing a pressure ulcer.

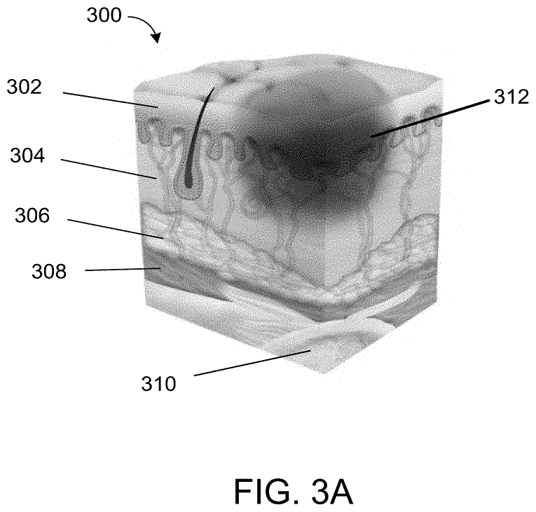

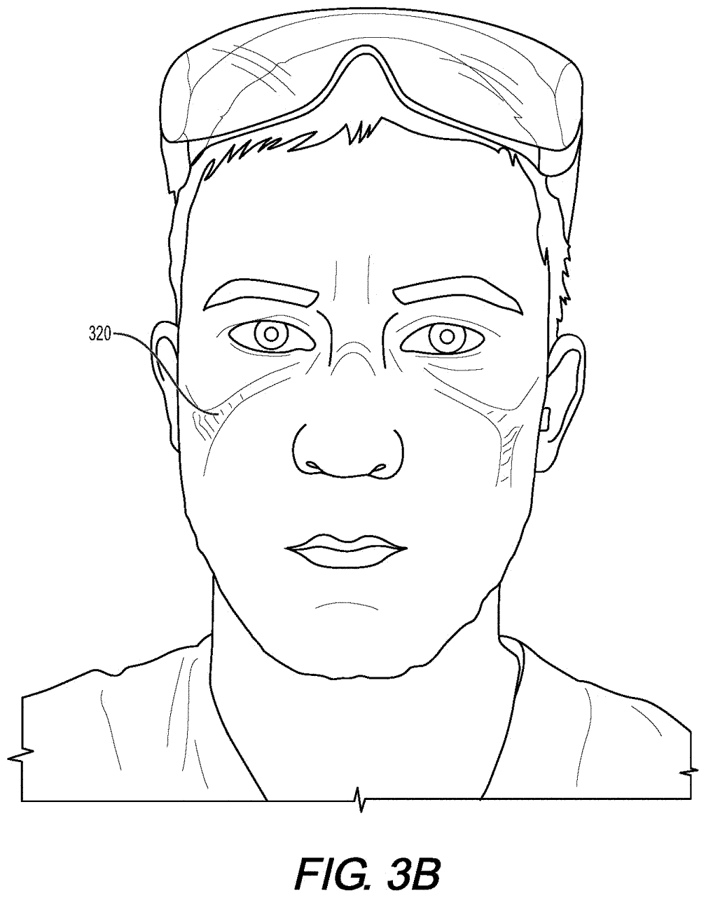

FIG. 3A illustrates the pressure-induced damage associated with a diagnosis of a stage-1 pressure ulcer. This cutaway view of a section of skin tissue 300 shows the top layer stratum corneum 302, the dermis 304, a layer of fat 306 over a layer of muscle 308, and a bone 310. The darkened region 312 indicates damage to the skin penetrating from the stratum corneum 302 down into the dermis 304. The surface of the skin over region 312 may show a redness and a difference in firmness that can be identified by a trained clinician as a symptom of the damage.

FIG. 3B depicts a user who has developed a pressure ulcer 320 from a PPE device worn on the face. Development of this type of injury depends on many factors, including the amount of local pressure on the skin, whether additional pressure was created by other items laying over the device, and the duration of the pressure. Development of an ulcer is also affected by the condition of the user's skin, which depends on the age of the user and their health.

FIG. 4A depicts a user 400 wearing a N95 mask 410 with a Sub-Epidermal Moisture (SEM) sensor (not visible in FIG. 4A), in accordance with the present disclosure. There is contact between the N95 mask 410 and the user 400 in multiple locations, such as the bridge of the nose 420, the cheeks 430, the chin 440. There is also contact between the elastic straps 450 holding the N95 mask 410 to the face, for example along the cheeks, above, below, and behind the ears 460. For the N95 mask to be effective in keeping out contaminants, a tight and complete seal between the mask and the face needs to be formed. This requires sufficient pressure at the areas where the N95 mask 410 contacts the face. As a result, prolonged usage of an N95 mask increases the risk of pressure injuries and pressure ulcers.

FIGS. 4B and 4C depicts an example SEM sensing system 470, in accordance with the present disclosure. The system 470 includes a PPE adaptor 422 (not shown) configured to attach to the edge of the N95 mask 410, a layer of foam 424 to distribute pressure, and a SEM sensor 431. In an aspect, there is a layer of adhesive 426 to attach the SEM sensing system to the skin of the user 400. The sensor 431 has electrodes 432, 434 that are connected via wires 436, 438 to electronics package 440, which is configured to make a measurement of the capacitance between the two electrodes 432, 434 and calculate a “delta” value that is, in one aspect, the difference between the highest SEM value and the lowest SEM value in a set of measurements. In an aspect, a set of measurements is taken during a single clinical evaluation. In one aspect, a set of measurements is taken over time, with the first measurement taken at the time of the first use of the PPE device.

In an aspect, a calculated delta value is compared to a threshold. When the delta value exceeds the threshold, this indicates a degree of damage. There may be multiple thresholds used to evaluate multiple levels of tissue damage. In one aspect, the maximum SEM value is compared to a threshold. When the maximum value exceeds the threshold, this indicates a degree of damage.

In an aspect, a threshold may be about 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5. In one aspect, a threshold may range from 0.1 to 8.0, such as from 0.1 to 1.0, from 1.1 to 2.0, from 2.1 to 3.0, from 3.1 to 4.0, from 4.1 to 5.0, from 5.1 to 6.0, from 6.1 to 7.0, from 7.1 to 8.0, from 0.1 to 7.5, from 0.5 to 8.0, from 1.0 to 7.0, from 1.5 to 6.5, from 2.0 to 6.0, from 3.0 to 5.5, from 3.5 to 5.0, or from 4.0 to 4.5. In an aspect, a threshold can be scaled by a factor or a multiple based on the values provided herein. It will be understood that a threshold is not limited by design, but rather, one of ordinary skill in the art would be capable of choosing a predetermined value based on a given unit of SEM. In one aspect, thresholds of the present disclosure are varied according to the specific portion of a user's body on which measurements are being made, or one or more characteristics of the user such as age, height, weight, family history, ethnic group, and other physical characteristics or medical conditions.

In an aspect, the electronics package 440 includes devices to communicate over link 442 to computer 452, which may be a PC, a mobile tablet, a mobile phone, a server using cloud-based data storage and analysis, or other data systems. Link 442 may include a wired or wireless communication element, optical communication elements, a network that may have one or more switches and routers, and other standard data transfer devices and protocols. Link 442 may also be implemented as hardware with nonvolatile storage, for example a “thumb drive,” that is loaded with data by the electronics package 440 and in turn is physically relocated and connected to the computer 452 whereupon it delivers the data. In an aspect, Link 442 provides real-time communication of recorded SEM measurements and/or calculated delta values from electronic package 440 to computer 452 to allow for real-time monitoring of ulcer development in a user.

In one aspect, a PPE adaptor 422 (not shown) of SEM sensing system 470 of the present disclosure is configured to attach to a PPE device selected from the group consisting of a respirator, a valved respirator, a full face respirator, a mask, a N95 mask, a KN95 mask, a face shield, safety helmet, goggles and ear muffs. In an aspect, adhesive 426 has a shape selected from the group consisting of substantially a square, substantially a rectangle, substantially a circle, and a polygon. In one aspect, a face of adhesive 426 has a surface area less than 25 cm², such as less than 20 cm², less than 15 cm², less than 10 cm², or less than 5 cm². In an aspect, SEM sensing system 450 has a mass of less than 5 grams, such as less than 4 grams, less than 3 grams, less than 2 grams, less than 1 gram, or less than 0.5 gram.

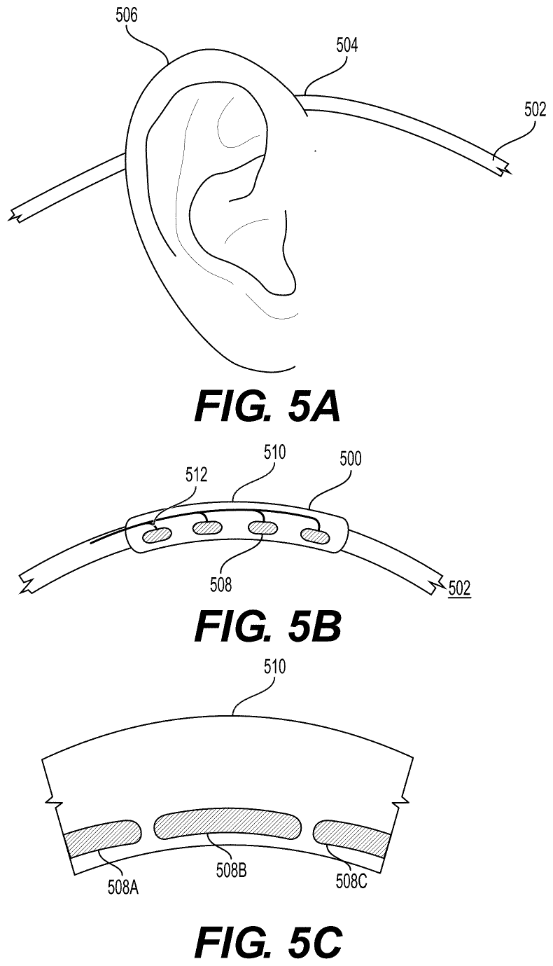

FIG. 5A illustrates how a PPE device may contact a user. An elastic strap 502 runs over the crease 504 between a user's ear 506 and their skull. Pressure can develop at the point of contact between elastic strap 502 and the crease 504 due to tension in elastic strap 502.

FIG. 5B depicts a SEM sensing device 500, in accordance with the present disclosure. In an aspect, the device 500 is added to a basic PPE device, for example elastic strap 502. Electrodes 508 on the external surface of the device body 510 are connected by wires 512 to an external electronics package (not shown in FIG. 5B). In an aspect, the device 500 comprises a processor (not visible in FIG. 5B) that does one or more of switching, sensing, and measurement. In an aspect, the processor provides wireless communication to the electronics package. In one aspect, the wireless communication to the electronics package from the electrodes occurs in real-time. In an aspect, the wireless communication to the electronics package is delayed.

FIG. 5C is an enlarged view of a portion of the device 500 of FIG. 5B, in accordance with the present disclosure. In this example, there are three electrodes 508A, 508B, and 508C that are aligned in a row on the surface of device body 510, but this array of electrodes may utilize two or more electrodes that are disposed in any two-dimensional pattern. In an aspect, device 500 may comprise three or more electrodes, such as four or more electrodes, five or more electrodes, ten or more electrodes, fifteen or more electrodes, twenty or more electrodes, twenty-five or more electrodes, thirty or more electrodes, forty or more electrodes, or fifty or more electrodes.

In FIG. 5C, electrodes 508A, 508B, 508C are elongated rectangles with rounded ends, but these electrodes may be provided in any shape and size. In an aspect, electrodes 508A, 508B, and 508C may be any shape or configuration, such as point electrodes, plate electrodes, ring electrodes, hexagonal electrodes, or interdigitated finger electrodes. In this example, the long, thin aspect ratio of the electrodes over the curved body 510 provides for complete contact between each electrode 508A, 508B, 508C and the user's skin. In one aspect, electrodes of device 500 are approximately evenly spaced apart by from about 0.1 cm to about 5 cm, such as from about 0.2 cm to about 5 cm, from about 0.3 cm to about 5 cm, from about 0.4 cm to about 5 cm, from about 0.5 cm to about 5 cm, from about 1 cm to about 5 cm, from about 1.5 cm to about 5 cm, from about 2 cm to about 5 cm, from about 2.5 cm to about 5 cm, from about 3 cm to about 5 cm, from about 3.5 cm to about 5 cm, from about 4 cm to about 5 cm, from about 4.5 cm to about 5 cm, from about 0.1 cm to about 4.5 cm, from about 0.1 cm to about 4 cm, from about 0.1 cm to about 3.5 cm, from about 0.1 cm to about 3 cm, from about 0.1 cm to about 2.5 cm, from about 0.1 cm to about 2 cm, from about 0.1 cm to about 1.5 cm, from about 0.1 cm to about 1 cm, from about 0.1 cm to about 0.9 cm, from about 0.1 cm to about 0.8 cm, from about 0.1 cm to about 0.7 cm, from about 0.1 cm to about 0.6 cm, from about 0.1 cm to about 0.5 cm, from about 0.1 cm to about 0.4 cm, from about 0.1 cm to about 0.3 cm, from about 0.1 cm to about 0.2 cm, from about 0.5 cm to about 4.5 cm, from about 1 cm to about 4 cm, from about 1.5 cm to about 3.5 cm, or from about 2 cm to about 3 cm. In an aspect, there is an insulating cover layer over each of the electrodes 508A, 508B, 508C.

Still referring to FIG. 5C, the electrodes 508A, 508B, 508C are individually coupled to the electronics package or other controlling processor such that pairs of any two electrodes may be selected to form a two-electrode sensor. With an array of electrodes, a plurality of sensors may be formed to measure capacitance over a region without moving the device 500.

For example, electrodes 508A, 508B can be paired to measure the SEM in the tissue between the electrodes 508A, 508B, then electrodes 508B, 508C can be paired to measure the SEM in the tissue between the electrodes 508B, 508C.

In an aspect, device 500 of the present disclosure is configured to attach to a PPE device selected from the group consisting of a respirator, a valved respirator, a full face respirator, a mask, a N95 mask, a KN95 mask, a face shield, safety helmet, goggles and ear muffs. In one aspect, device 500 has a mass of less than 5 grams, such as less than 4 grams, less than 3 grams, less than 2 grams, less than 1 gram, or less than 0.5 gram.

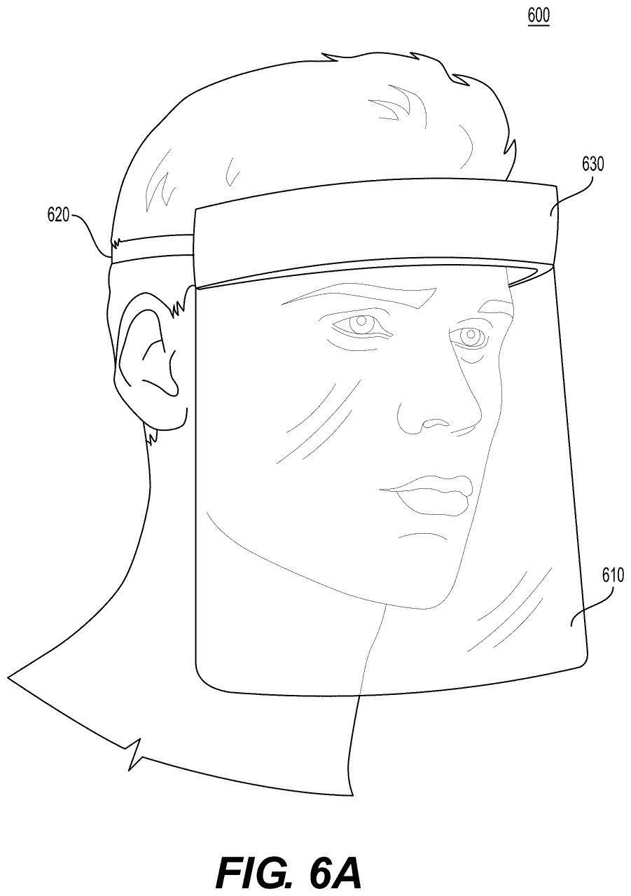

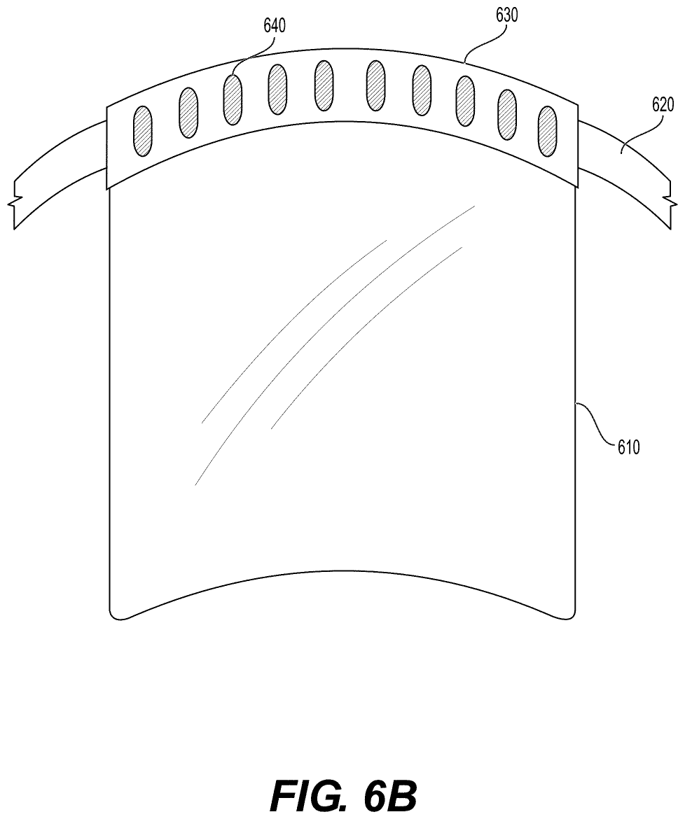

FIG. 6A depicts a user 600 wearing a face shield 610 that incorporates an elastic strap 620 to hold the face shield 610 in place via the forehead band 630, in accordance with the present disclosure. The forehead band 630 is held in place by contact with the forehead. Prolonged use of a face shield increases the risk of tissue damage and pressure ulcers formed due to the contact at the forehead.

FIG. 6B is an enlarged view of the underside of the forehead band 630 of FIG. 6A, in accordance with the present disclosure. In this example, electrodes 640 are attached to the forehead band 630 such that the electrodes 640 are in contact with the user's skin while the face shield 610 is worn. In one aspect, electrodes 640 are elongated-shaped electrodes. In an aspect, similar electrodes (not shown in FIG. 6B) are located on the contact surface of the elastic strap 620. As described with respect to FIG. 5C, the individual electrodes of an array of electrodes 640 can be connected in various pairs to form sensors. In an aspect, the forehead band 630 includes one or more of a battery, a processor, data storage, and a communication element.

In an aspect, forehead band 630 may comprise two or more electrodes, such as three or more electrodes, four or more electrodes, five or more electrodes, ten or more electrodes, fifteen or more electrodes, twenty or more electrodes, twenty-five or more electrodes, thirty or more electrodes, forty or more electrodes, fifty or more electrodes or a hundred or more electrodes.

In one aspect, electrodes of forehead band 630 are approximately evenly spaced apart by from about 0.1 cm to about 5 cm when the retention strap is in a relaxed state, such as from about 0.2 cm to about 5 cm, from about 0.3 cm to about 5 cm, from about 0.4 cm to about 5 cm, from about 0.5 cm to about 5 cm, from about 1 cm to about 5 cm, from about 1.5 cm to about 5 cm, from about 2 cm to about 5 cm, from about 2.5 cm to about 5 cm, from about 3 cm to about 5 cm, from about 3.5 cm to about 5 cm, from about 4 cm to about 5 cm, from about 4.5 cm to about 5 cm, from about 0.1 cm to about 4.5 cm, from about 0.1 cm to about 4 cm, from about 0.1 cm to about 3.5 cm, from about 0.1 cm to about 3 cm, from about 0.1 cm to about 2.5 cm, from about 0.1 cm to about 2 cm, from about 0.1 cm to about 1.5 cm, from about 0.1 cm to about 1 cm, from about 0.1 cm to about 0.9 cm, from about 0.1 cm to about 0.8 cm, from about 0.1 cm to about 0.7 cm, from about 0.1 cm to about 0.6 cm, from about 0.1 cm to about 0.5 cm, from about 0.1 cm to about 0.4 cm, from about 0.1 cm to about 0.3 cm, from about 0.1 cm to about 0.2 cm, from about 0.5 cm to about 4.5 cm, from about 1 cm to about 4 cm, from about 1.5 cm to about 3.5 cm, or from about 2 cm to about 3 cm.

In an aspect, forehead band 630 of the present disclosure is configured to attach to a visor. In one aspect, forehead band 630 of the present disclosure is configured to attach to safety goggles.

In one aspect, a face of forehead band 630 has a surface area less than 6000 cm², such as less than 5000 cm², less than 4000 cm², less than 3000 cm², less than 2000 cm², less than 1000 cm², less than 500 cm², less than 100 cm², less than 50 cm², less than 25 cm², less than 20 cm², less than 15 cm², less than 10 cm², or less than 5 cm².

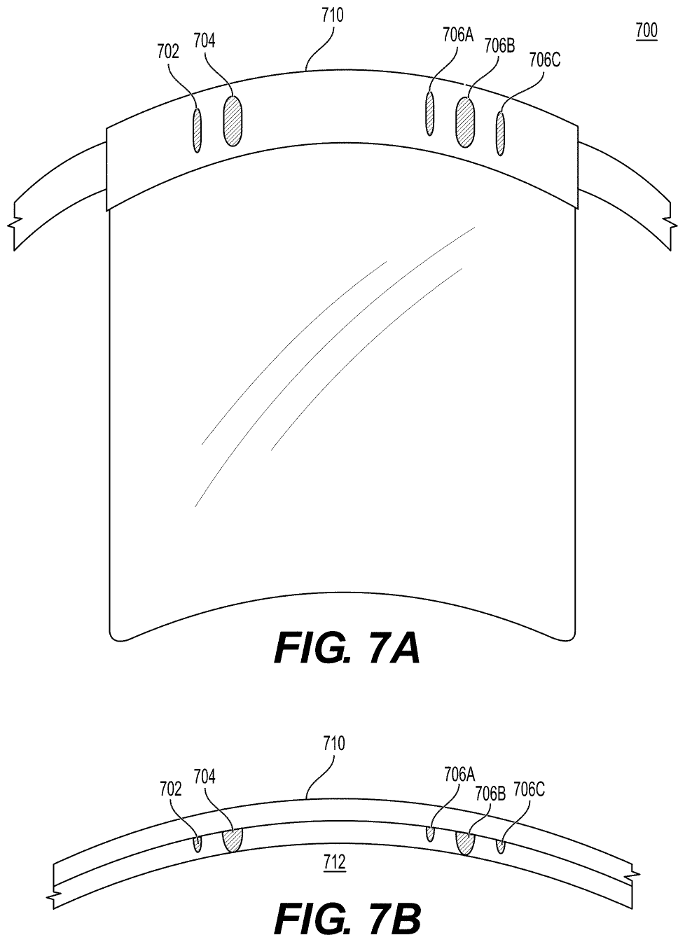

FIG. 7A depicts an example PPE device 700 with controllable pressure management elements, in accordance with the present disclosure. FIG. 7B depicts the top view of the headband 710 where it contacts the forehead 712 of a user. In this example, the PPE device 700 is a face shield that is representative of all devices where the controllable pressure management element is in long-term contact with the skin of a user. In an aspect, a PPE device having a controllable pressure management element in long-term contact with the skin of a user is an N95 mask. In one aspect, the pressure management element is located on the ear straps of a mask. In one aspect, a PPE device having a controllable pressure management element in long-term contact with the skin of a user is a respirator. In one aspect, a PPE device having a controllable pressure management element in long-term contact with the skin of a user is safety goggles. In this example, the pressure management elements are inflatable pockets such as pocket 702, which is shown in an inactive, e.g., deflated, state. Pocket 704, by way of comparison, is shown in an active, e.g., inflated, state. When pockets 702, 704 are configured as shown in FIG. 7A, pressure is higher in the region of pocket 704 and lower in the region of pocket 702. In an aspect, the pressure in the region of pocket 702 is low enough to allow blood flow through the tissue of this region.

In an aspect, the pressure management elements are provided in sets such as pockets 706A, 706B, and 706C. These pockets may be manipulated in a coordinated fashion to shift the levels of contact pressure between the device 700 and the skin of the user in the regions of the pockets 706A, 706B, 706C. For example, the pocket 706B is inflated while pockets 706A, 706C are deflated, creating a relatively high contact pressure area around pocket 706B and a relatively low, e.g. lower than the nominal pressure that would be present in the absence of a pressure management element, contact pressure in the regions of pockets 706A, 706C. This relatively low contact pressure allows adequate blood flow to the tissue in that region so as to avoid tissue damage. At a different time, one or both of pockets 706A, 706C are inflated while pocket 706B is deflated, thus reducing the contact pressure in the region of pocket 510B.

In an aspect, the pockets are flexible membranes that comprise a portion of the walls of a sealed compartment that is within or on the surface of device 700. In an aspect, at least one of the walls of the pockets is stretchable. In one aspect, when the pockets are situated within the surface of device 700, the wall of device 700 that is in contact with the skin of a user is also stretchable.

The words “force” and “pressure” are considered to be interchangeable within the context of this disclosure. A higher pressure within a pocket will apply a greater pressure over the area of the pocket, which produces a higher total force (pressure×area=force). A greater amount of fluid in the pocket does not intrinsically apply a higher pressure or force; the raised height of the pocket will cause the user's skin to come in contact with the inflated pocket first and thereby the inflated pocket will provide a greater portion of the total force applied by the device 700 to the user's skin and such is equivalent to providing a greater pressure and/or force.

Pockets may be fully inflated, fully deflated, or partially inflated to an intermediate pressure. In an aspect, the pockets may be inflated with a gas or a liquid or other fluid. The word “inflation” is interpreted as an indication of pressure or, equivalently, of the amount of fluid within the pocket, such that the phrase “higher inflation” includes the situation of a greater amount of fluid in the compartment.

In an aspect, the pockets are connected to a source of pressurized fluid through elements such as tubing, valves, pressure regulators (not shown in FIG. 7A) that are coupled to and controlled by a controller (not shown in FIG. 7A). In an aspect, the source of pressurized fluid may be the same source of fluid being provided to the user through the PPE device 700, for example pressurized oxygen-enriched air. In an aspect, the controller of the pressure management element is a part of the electronics package 440 of FIG. 4C.

In an aspect, the pressure management element is a mechanical element whose height can be adjusted. In an aspect, the adjustment is provided with an electrical actuator. In an aspect, the actuator comprises a piezoelectric element that causes a change in the height of the element. In an aspect, the pressure management element is a fixed height element that moves parallel to the skin of the user such that the contact pressure is increased in the region of contact between the element and the skin and reduced in other regions.

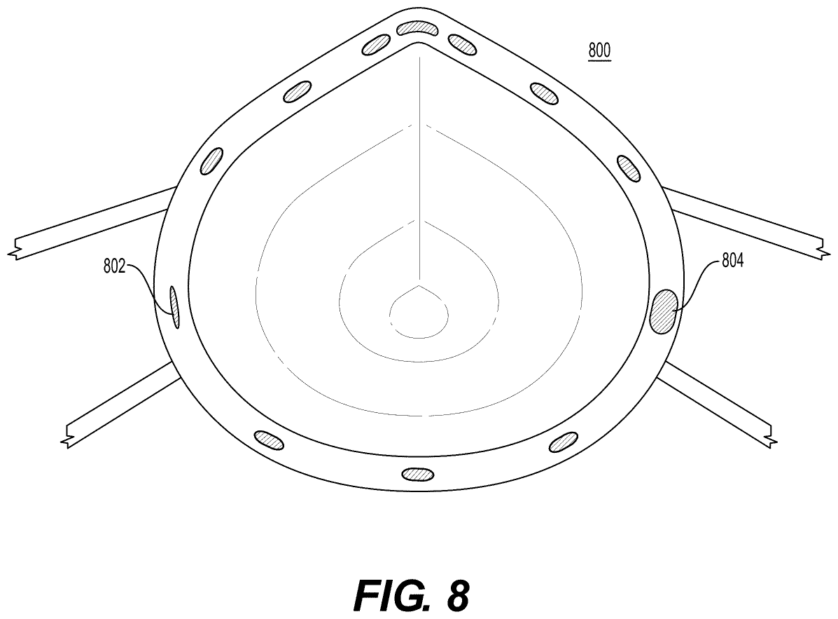

FIG. 8 illustrates another PPE device that is a PPE device 800 similar to the N95 mask 410 shown in FIGS. 4A and 4B. In this example, the PPE device 800 comprises a pressure management element with pockets such as pockets 802, 804 spaced along the edge of the PPE device 800. In this example, pocket 802 is inactive and pocket 804 is active, causing the contact pressure under pocket 804 to be higher than the contact pressure under pocket 802. In an aspect, the edge of PPE device 800 is overlaid with an array of SEM sensors 431 comprising electrodes 432 and 434 (not visible in FIG. 7B) such that PPE device 800 can both measure SEM and manage the pressure applied by the PPE device 800 to the user's skin.

In an aspect, the change in inflation of the pockets is driven by an SEM reading taken, for example, by the electrodes 640 of FIG. 6B. In one aspect, the change in inflation of the pockets is driven by a delta value that is, in an aspect, the difference between the highest SEM value and the lowest SEM value in a set of measurements. In an aspect, a set of measurements includes measurements taken at a single location. In one aspect, a set of measurements includes measurements taken at multiple locations. In one aspect, a set of measurements is taken at approximately the same time, such as within 10 minutes, within 5 minutes, within 1 minute, within 30 seconds, within 10 seconds, within 5 seconds, or within 1 second. In an aspect, a delta value is calculated by the difference between the most recent SEM value and the cumulative average SEM value over a period of time. In one aspect, a cumulative average SEM value is derived from a set of SEM measurements taken since the first use of the PPE device. In an aspect, a cumulative average SEM value is derived from SEM measurements taken within approximately a year, such as within 9 months, within 6 months, within 5 months, within 4 months, within 3 months, within 2 months, within 1 month, within four weeks, within three weeks, within two weeks, within one week, within 6 days, within 5 days, within 4 days, within 3 days, within 2 days, within 1 day, within 16 hours, within 12 hours, within 8 hours, within 4 hours, within 3 hours, within 2 hours, within 1 hour, within 45 minutes, within 30 minutes, within 15 minutes, within 10 minutes, or within 5 minutes.

In an aspect, the change in inflation of the pockets is driven by how a calculated delta value is compared to a threshold. When the delta value exceeds the threshold, inflation pattern of the pockets changes to shift the pressure applied to the users. There may be multiple thresholds used to determine the inflation pattern of the pockets.

In an aspect, the change in inflation is caused by a timer that regularly shifts the pressure applied to the user by changing the pattern of active pressure management elements, for example by inflating and deflating different pockets.

In an aspect, a series of predetermined configurations of the pressure management elements are defined and the timer configured to execute a programmed series of changes between these configurations at predefined times. In an aspect, the changes between predetermined configurations are based on SEM readings taken of the user.

Without being bound by theory, constant adjustments of PPE devices, e.g., touching, tightening, or loosening, increases the risk of device failure or contamination. Prolonged usage of PPE devices, e.g., during long work shifts, may increase the discomfort of the user, and the ability to adjust the PPE device without touching it may increase user safety. In an aspect, the change in inflation is controlled by the user remotely. In an aspect, the user can activate or deactivate the pressure management elements of the PPE device without touching the PPE device. In an aspect, the user can inflate the pockets to enable a better seal when needed, e.g., when the user performing an intubation procedure, or in an area with high concentrations of a contaminant. In an aspect, the user can deflate the pockets to enable to reduce pressure on the skin when needed, e.g., when the user is taking a break or in an area with low concentrations of a contaminant.

In an aspect, there is a configuration of which pockets are inflated and this default is maintained until a SEM reading indicates a problem, whereupon certain pockets are deflated or reduced in inflation height.

From the foregoing, it will be appreciated that the present invention can be embodied in various ways, which include but are not limited to the following:

Embodiment 1. An apparatus for detecting tissue damage proximate to a point of contact between a PPE device and a user's skin, comprising: a first electrode and a second electrode configured to measure a level of sub-epidermal moisture (SEM) in tissue proximate to the point of contact, an electronics package individually connected to the first and second electrodes and configured to measure a capacitance between the first and second electrodes.

Embodiment 2. The apparatus of embodiment 1, where the first and second electrodes are configured to be attached to the PPE device.

Embodiment 3. The apparatus of embodiment 1 or 2, where the first and second electrodes are shaped such that the entire surface of each electrode can contact the user's skin while the PPE device is in use.

Embodiment 4. The apparatus of any one of embodiments 1 to 3, further comprising a body coupled to the first and second electrodes, the body configured to be interposed between the PPE device and the user's skin when the PPE device is in use.

Embodiment 5. The apparatus of embodiment 4, where the body is further configured to be attached to the PPE device.

Embodiment 6. The apparatus of any one of embodiments 1 to 5, further comprising a communication element configured to provide real-time transfer of SEM measurements to a computing unit.

Embodiment 7. The apparatus of any one of embodiments 1 to 6, where the apparatus is an adaptor configured to attach to an edge of the PPE device.

Embodiment 8. The apparatus of any one of embodiments 1 to 6, where the apparatus is configured to attach to a forehead band of a face shield.

Embodiment 9. The apparatus of any one of embodiments 1 to 6, where the apparatus is configured to attach to the edge of a mask.

Embodiment 10. The apparatus of embodiment 9, where the PPE device is an N95 mask.

Embodiment 11. The apparatus of any one of embodiments 1 to 6, where the PPE device is selected from the group consisting of a respirator, a valved respirator, a full face respirator, a mask, a N95 mask, a KN95 mask, a face shield, safety helmet, goggles and ear muffs.

Embodiment 12. The apparatus of any one of embodiments 1 to 11, where the apparatus further comprises one or more pressure management elements.

Embodiment 13. The apparatus of embodiment 12, where each of the one or more pressure management elements is an inflatable pocket.

Embodiment 14. A method for detecting tissue damage proximate to a point of contact between a PPE device and a user's skin, comprising the steps of: measuring a plurality of sub-epidermal moisture (SEM) values of tissue proximate to the point of contact at incremental times, comparing the plurality of SEM values, and determining if there is a significant increase in the SEM that indicates that there is tissue damage.

Embodiment 15. The method of embodiment 14, where there is a significant increase when the largest SEM value of the plurality of SEM values is greater than the smallest SEM value of the plurality of SEM values by an amount that exceeds a threshold.

Embodiment 16. The method of embodiment 14, where there is a significant increase when the largest SEM value of the plurality of SEM values is greater than a threshold.

Embodiment 17. The method of any one of embodiments 14 to 16, where a first measurement of the SEM value is made at the time of the first use of the PPE device.

Embodiment 18. The method of any one of embodiments 14 to 17, where the PPE device is selected from a group consisting of a respirator, a valved respirator, a full face respirator, a mask, a N95 mask, a KN95 mask, a face shield, safety helmet, goggles and ear muffs.

Embodiment 19. The method of any one of embodiments 14 to 17, where the PPE device is a mask.

Embodiment 20. The method of any one of embodiments 14 to 17, where the PPE device is a face shield.

We claim:

1. An apparatus for detecting tissue damage proximate to a point of contact between a PPE device and a user's skin, comprising:
a first electrode and a second electrode configured to measure a level of sub-epidermal moisture (SEM) in tissue proximate to the point of contact,
an electronics package individually connected to the first and second electrodes and configured to measure a capacitance between the first and second electrodes.

2. The apparatus of claim 1, wherein the first and second electrodes are configured to be attached to the PPE device.

3. The apparatus of claim 1, wherein the first and second electrodes are shaped such that the entire surface of each electrode can contact the user's skin while the PPE device is in use.

4. The apparatus of claim 1, further comprising a body coupled to the first and second electrodes, the body configured to be interposed between the PPE device and the user's skin when the PPE device is in use.

5. The apparatus of claim 4, wherein the body is further configured to be attached to the PPE device.

6. The apparatus of claim 1, further comprising a communication element configured to provide real-time transfer of SEM measurements to a computing unit.

7. The apparatus of claim 1, wherein the apparatus is a PPE adaptor configured to attach to the edge of the PPE device.

8. The apparatus of claim 1, wherein the apparatus is configured to attach forehead band of a face shield.

9. The apparatus of claim 1, wherein the apparatus is configured to attach to the edge of a mask.

10. The apparatus of claim 9, wherein the PPE device is an N95 mask.

11. The apparatus of claim 1, wherein the PPE device is selected from the group consisting of a respirator, a valved respirator, a full face respirator, a mask, a N95 mask, a KN95 mask, a face shield, safety helmet, goggles and ear muffs.

12. The apparatus of claim 1, wherein the apparatus further comprises one or more pressure management elements.

13. The apparatus of claim 12, wherein each of the one or more pressure management elements is an inflatable pocket.

14. A method for detecting tissue damage proximate to a point of contact between a PPE device and a user's skin, comprising the steps of:
measuring a plurality of sub-epidermal moisture (SEM) values of tissue proximate to the point of contact at incremental times,
comparing the plurality of SEM values, and
determining if there is a significant increase in the SEM that indicates that there is tissue damage.

15. The method of claim 14, wherein there is a significant increase when the largest SEM value of the plurality of SEM values is greater than the smallest SEM value of the plurality of SEM values by an amount that exceeds a threshold.

16. The method of claim 14, wherein there is a significant increase when the largest SEM value of the plurality of SEM values is greater than a threshold.

17. The method of claim 14, wherein a first measurement of the SEM value is made at the time of the first use of the PPE device.

18. The method of claim 14, wherein the PPE device is selected from the group consisting of a respirator, a valved respirator, a full face respirator, a mask, a N95 mask, a KN95 mask, a face shield, safety helmet, goggles and ear muffs.

19. The method of claim 14, wherein the PPE device is a mask.

20. The method of claim 14, wherein the PPE device is a face shield.
